(19) United States
(12) Patent Application Publication
(10) Pub. No.: US 2023/0190919 A1
(43) Pub. Date: Jun. 22, 2023

(54) CORONAVIRUS VACCINE COMPOSITIONS AND METHODS OF USE

(71) Applicant: Thomas Jefferson University, Philadelphia, PA (US)

(72) Inventors: Douglas Craig Hooper, Medford, NJ (US); Bernhardt Dietzschold, Philadelphia, PA (US)

(21) Appl. No.: 17/996,119

(22) PCT Filed: Apr. 13, 2021
(86) PCT No.: PCT/US2021/027017

§ 371 Date: Oct. 13, 2022

Related U.S. Application Data
(60) Provisional application No. 63/009,131, filed on Apr. 13, 2020.

(51) Int. Cl. A61K 39/215; C07K 14/005; A61K 39/205

(52) U.S. Cl. CPC A61K 39/215; A61K 39/205; C07K 14/005; A61K 2039/5254; C12N 2760/20121; C12N 2760/20122; C12N 2760/20134; C12N 2760/20162; C12N 2760/20171; C12N 2770/20022; C12N 2770/20034; C12N 2770/20071

(57) ABSTRACT

Provided is a method of enhancing an antigen-induced long-lasting immune response in a host comprising administering to a host an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen that is not expressed by the rabies virus. Also provided are related compositions, kits and vaccines.

### STATEMENT REGARDING FEDERALLY SPONSORED RESEARCH OR DEVELOPMENT

This invention was made with government support under AI093369 and AI093666 awarded by the National Institute of Health. The government has certain rights in the invention.

### CROSS-REFERENCE TO RELATED APPLICATIONS

This application claims priority to U.S. Provisional Application No. 63/009,131, filed Apr. 13, 2020, the disclosure of which is hereby incorporated by reference in its entirety.

### FIELD OF THE INVENTION

The invention relates to the field of biotechnology and immunology, and in particular to the utilization of recombinant attenuated rabies virus to promote the development of immunity to co-administered antigens of a Coronavirus, such as severe acute respiratory syndrome coronavirus 2 (SARS-CoV-2).

### BACKGROUND OF THE INVENTION

Coronavirus disease 2019 (COVID-19) is an inflammatory lung disease caused by infection with the severe acute respiratory syndrome coronavirus 2 (SARS-CoV-2) / 2019 novel coronavirus (2019-nCoV) virus. Infection with the virus most often does not cause severe disease but can cause lethal lung inflammation in some individuals suggesting an immune contribution to the disease. This highlights the requirement for SARS-CoV-2 vaccine strategies biased towards the production of neutralizing antibodies. However, no SARS-CoV-2 vaccine is available yet.

Thus, there is a need in the art for improved compositions and methods to treat and prevent SARS-CoV-2 infection. This invention satisfies this unmet need.

### SUMMARY OF THE INVENTION

In one aspect, the present invention relates, in part, to a method of inducing an immune response against a coronavirus in a subject. In various embodiments, the method of the present invention comprises administering to the subject an effective amount of: (a) a nonpathogenic recombinant rabies virus comprising at least three copies of a mutated G gene; and (b) a Coronavirus antigen, or a nucleic acid molecule encoding a Coronavirus antigen.

In one embodiment, the recombinant rabies virus does not express a foreign protein antigen.

In one embodiment, the mutated G gene encodes a rabies virus glycoprotein. In one embodiment, the glycoprotein amino acid 194 is serine and the glycoprotein amino acid 333 is glutamic acid.

In one embodiment, the Coronavirus antigen is not expressed by the rabies virus. In some embodiments, the Coronavirus antigen comprises an antigen of a Coronavirus selected from the group consisting of: SARS-CoV-2, SARS-CoV, MERS-CoV, 229E, NL63, OC43, and HKU1. In some embodiments, the Coronavirus antigen comprises SARS-CoV-2 Spike (S) protein or fragment thereof. In one embodiment, the Coronavirus antigen comprises a fragment of SARS-CoV-2 S that binds to angiotensin-converting enzyme 2.

In some embodiments, the nonpathogenic recombinant rabies virus and the Coronavirus antigen, or nucleic acid molecule encoding the Coronavirus antigen, are administered simultaneously. In some embodiments, the nonpathogenic recombinant rabies virus is administered before the Coronavirus antigen, or nucleic acid molecule encoding the Coronavirus antigen.

In some embodiments, the nonpathogenic recombinant rabies virus and the Coronavirus antigen, or nucleic acid molecule encoding the Coronavirus antigen, are administered in the same composition. In some embodiments, the nonpathogenic recombinant rabies virus and the Coronavirus antigen, or nucleic acid molecule encoding the Coronavirus antigen, are administered in a different composition.

In some embodiments, the nonpathogenic recombinant rabies virus and the Coronavirus antigen, or nucleic acid molecule encoding the Coronavirus antigen, are administered intranasally, subcutaneously, epidermaly, intradermally, intrathecally, intraorbitally, intramucosally, intraperitoneally, intravenously, orally, or intramuscularly.

In one aspect, the present invention relates, in part, to a composition comprising: (a) a nonpathogenic recombinant rabies virus comprising at least three copies of a mutated G gene, wherein said mutated G gene encodes a rabies virus glycoprotein; and (b) a Coronavirus antigen, or a nucleic acid molecule encoding a Coronavirus antigen. In various embodiments, the composition comprises (a) a nonpathogenic recombinant rabies virus comprising at least three copies of a mutated G gene encoding a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen, or a nucleic acid molecule encoding a Coronavirus antigen, wherein the Coronavirus antigen is not expressed by the rabies virus.

In one embodiment, the Coronavirus antigen is not expressed by the rabies virus. In some embodiments, the Coronavirus antigen comprises an antigen of a Coronavirus selected from the group consisting of: SARS-CoV-2, SARS-CoV, MERS-CoV, 229E, NL63, OC43, and HKU1. In some embodiments, the Coronavirus antigen comprises SARS-CoV-2 Spike (S) protein or fragment thereof. In one embodiment, the Coronavirus antigen comprises a fragment of SARS-CoV-2 S that binds to angiotensin-converting enzyme 2.

In various embodiments, the composition of the present invention is a vaccine.

In one aspect, the present invention relates, in part, to a method of administering the composition of the present invention to a subject. In one embodiment, the subject has a severe acute respiratory syndrome coronavirus 2 (SARS-CoV-2).

In one aspect, the present invention relates, in part, to a kit for immunization of a subject comprising: (a) a nonpathogenic recombinant rabies virus comprising at least three copies of a mutated G gene, wherein said mutated G gene encodes a rabies virus glycoprotein; and (b) a Coronavirus antigen, or a nucleic acid molecule encoding a Coronavirus antigen. In various embodiments, the kit comprises (a) a nonpathogenic recombinant rabies virus comprising at least three copies of a mutated G gene encoding a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen, or a nucleic acid molecule encoding a Coronavirus antigen, wherein the Coronavirus antigen is not expressed by the rabies virus.

In one embodiment, the Coronavirus antigen is not expressed by the rabies virus. In some embodiments, the Coronavirus antigen comprises an antigen of a Coronavirus selected from the group consisting of: SARS-CoV-2, SARS-CoV, MERS-CoV, 229E, NL63, OC43, and HKU1. In some embodiments, the Coronavirus antigen comprises SARS-CoV-2 Spike (S) protein or fragment thereof. In one embodiment, the Coronavirus antigen comprises a fragment of SARS-CoV-2 S that binds to angiotensin-converting enzyme 2.

In one aspect, the present invention relates, in part, to a method of immunizing a subject against a Coronavirus antigen comprising the steps of: (a) administering a first composition comprising a nonpathogenic recombinant rabies virus comprising at least three copies of a mutated G gene; and (b) administering a second composition comprising a Coronavirus antigen, or a nucleic acid molecule encoding a Coronavirus antigen.

In another aspect, the present invention relates, in part, to a method of immunizing a subject against rabies virus and against a Coronavirus antigen comprising the steps of: (a) administering a first composition comprising a nonpathogenic recombinant rabies virus comprising at least three copies of a mutated G gene; and (b) administering a second composition comprising a Coronavirus antigen, or a nucleic acid molecule encoding a Coronavirus antigen.

In various embodiments, the method comprises (a) administering a first composition comprising a nonpathogenic recombinant rabies virus comprising at least three copies of a mutated G gene encoding a rabies virus glycoprotein wherein the glycoprotein amino acid 194 is serine and the glycoprotein amino acid 333 is glutamic acid, wherein said recombinant rabies virus does not express a foreign protein antigen; and (b) administering a second composition comprising a Coronavirus antigen, or a nucleic acid molecule encoding a Coronavirus antigen, wherein the Coronavirus antigen is not expressed by the rabies virus.

In one embodiment, the Coronavirus antigen is not expressed by the rabies virus. In some embodiments, the Coronavirus antigen comprises an antigen of a Coronavirus selected from the group consisting of: SARS-CoV-2, SARS-CoV, MERS-CoV, 229E, NL63, OC43, and HKU1. In some embodiments, the Coronavirus antigen comprises SARS-CoV-2 Spike (S) protein or fragment thereof. In one embodiment, the Coronavirus antigen comprises a fragment of SARS-CoV-2 S that binds to angiotensin-converting enzyme 2.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A and FIG. 1B, depicts representative results demonstrating the production of ovalbumin (OVA)-specific antibody after immunization with a single dose of a mixture of TriGAS and OVA or OVA alone. The titers are presented as mean titers (+/- Standard Error) calculated from ten mice per group. Groups of ten Swiss Webster mice were immunized intramuscularly (i.m.) with 100 µL PBS containing 10⁶ FFU TriGAS and 50 µg OVA (open bars) or 100 µL PBS containing only 50 µg OVA (closed bars). The isotypes of OVA-specific antibody were determined by ELISA using alkaline-phosphatase conjugated antimouse whole IgG or biotinylated anti-mouse IgG1, IgG2a, or IgG2b. FIG. 1A depicts representative results demonstrating the production of ovalbumin (OVA)-specific antibody after immunization with a single dose of a mixture of TriGAS and OVA or OVA alone. The mice were bled 21 days after immunization. FIG. 1B depicts representative results demonstrating the production of ovalbumin (OVA)-specific antibody after immunization with a single dose of a mixture of TriGAS and OVA or OVA alone. The mice were bled 2 months after immunization.

FIG. 2, comprising FIG. 2A and FIG. 2B, depicts representative results demonstrating the production of Nipah virus glycoprotein (NiVG)-specific antibody produced after immunization with a single dose of a mixture of TriGAS and NiVG or NiVG alone. Groups of ten Swiss Webster mice were immunized i.m. with 100 µL PBS containing 10⁶ FFU TriGAS and 12.5 µg NiVG (open bars), or 100 µL PBS containing only 12.5 µg NiVG (closed bars). The isotypes of NiVG-specific antibody were determined by ELISA using alkalinephosphatase conjugated anti-mouse whole IgG or biotinylated anti-mouse IgG1, IgG2a, or IgG2b. The titers are presented as mean titers (+/- Standard Error) calculated from ten mice per group. FIG. 2A depicts representative results demonstrating the production of Nipah virus glycoprotein (NiVG)-specific antibody produced after immunization with a single dose of a mixture of TriGAS and NiVG or NiVG alone. The mice were bled 21 days after immunization. FIG. 2B depicts representative results demonstrating the production of Nipah virus glycoprotein (NiVG)-specific antibody produced after immunization with a single dose of a mixture of TriGAS and NiVG or NiVG alone. The mice were bled 2 months after immunization.

FIG. 3 depicts representative results demonstrating the production of Gonadotropin releasing hormone (GnRH)-specific antibody produced after immunization with a single dose of a mixture of TriGAS and GnRH-keyhole limpet hemocyanin (KLH), or with GnRH-KLH or TriGAS alone. Groups of ten Swiss Webster mice were immunized intramuscularly (i.m.) with 100 µL PBS containing 10⁶ FFu TriGAS and 50 µg GnRH (as GnRH-KLH), or 100 mL PBS containing only GnRH-KLH (50 µg GnRH). The mice were bled 21 days, 3 months, and 6 months after immunization and GnRH-specific antibody were determined by ELISA using alkaline phosphatase-labelled anti-mouse whole IgG. The titers are presented as mean titers (+/- Standard Error) calculated from ten mice per group.

### DETAILED DESCRIPTION

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, New York; Houston et al., 1988, Proc. Natl. Acad. Sci. USA 85:5879- 5883; Bird et al., 1988, Science 242:423-426).

As used herein, each “amino acid” is represented by the full name thereof, by the three letter code corresponding thereto, or by the one-letter code corresponding thereto, as indicated in the following table:

| | | |
|---|---|---|
| Full Name | Three-Letter Code | One-Letter Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids.

The term “animal” has its ordinary meaning, and is meant to include human beings.

“Attenuated” as used herein in the context of a live virus, such as a rabies virus, means that the ability for the virus to infect a cell or subject and/or its ability to produce disease is reduced (for example, eliminated). Attenuation may arise from either a genetic mechanism involving premature termination of transcription from the RV genome, or immunologically as a process whereby a pathogenic RV loses its virulence.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Gene expression” or “expression” as used herein refers to the process by which information from a gene is made into a functional gene product, such as RNA or protein. Thus, the “level of expression” of a gene product of a marker gene of the, in a sample of interest, refers to the level of RNA, particularly the level of mRNA, or the level of the encoded protein, and is not intended to be limited to either.

As used herein, an “expression vector” is a genetic element that functions as an autonomous unit of DNA replication under its own control sequence, to which another DNA segment may be attached or inserted so as to bring about replication of the attached or inserted segment. Expression vectors include plasmids, phages or cosmids. In general, expression vectors contain promoter sequences which facilitate the efficient transcription and translation of the attached or inserted DNA segment in a particular host cell. The expression vector also typically contains an origin of replication and transcription terminator(s), as well as specific genes which are capable of providing phenotypic selection in transfected host cells

By a “foreign protein antigen” with respect to a recombinant rabies virus is meant an antigen of a protein that is not native to the rabies virus expressing the antigen. In certain embodiments where the foreign antigen is an antigen of a virus, the virus is other than a rabies virus.

By a “Coronavirus antigen” is meant an antigen of a protein that is a Coronavirus protein. In certain embodiments, the Coronavirus antigen comprises a protein, protein subunit, domain, subdomain, or antigenic fragment of a Coronavirus protein. In some embodiments, the Coronavirus antigen is conjugated to a carrier such as keyhole limpet hemocyanin (KLH).

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. [0048] As used herein, “gene products” include any product that is produced in the course of the transcription, reverse -transcription, polymerization, translation, post-translation and/or expression of a gene. Gene products include, but are not limited to, proteins, polypeptides, peptides, peptide fragments, or polynucleotide molecules.

As used herein, “RV G protein gene” or “G protein gene” or “G gene” means the nucleic acid sequences which, when present in an RV genome, are sufficient to encode an RV glycoprotein in an infected cell. The G protein gene thus includes a coding sequence which is flanked on the 3′ end with a short transcriptional start sequence, and on the 5′ end with a short transcriptional stop/polyadenylation sequence. The G gene can include an intergenic region of 1-59 untranslated nucleotides, which is located between the 5′ stop/polyadenylation sequence and the 3′ transcriptional start sequence of the next viral gene. See, e.g., pgs. 134- 136 of Conzelman K-K (1998), Ann. Rev. Genet. 32: 123-62.

The term “immunization” refers to a process to induce partial or complete protection against a disease. Alternatively, the term refers to a process to induce or amplify an immune system response to an antigen.

An “immune response” to an antigen or vaccine composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell- mediated immune response” is elicited by the presentation of antigenic epitopes in association with Class I or Class II molecules of the major histocompatability complex (MHC).

A “mutation,” as used herein, refers to a change in nucleic acid or polypeptide sequence relative to a reference sequence (such as a naturally-occurring normal or “wild-type” sequence), and includes translocations, deletions, insertions, and substitutions/point mutations. A “mutant,” as used herein, refers to either a nucleic acid or protein comprising a mutation.

A “nucleic acid” refers to a polynucleotide and includes poly-ribonucleotides and poly-deoxyribonucleotides.

A coding sequence is “operably linked” to a control sequence such as transcriptional and translational control sequence in a cell when RNA polymerase transcribes the coding sequence into mRNA, which is then trans-RNA spliced and translated into the protein encoded by the coding sequence.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, anti- sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications.

As used herein, “pharmaceutical compositions” include formulations for human and veterinary use.

As used herein, “promoter” refers to a region of a DNA sequence active in the initiation and regulation of the expression of a structural gene. This sequence of DNA, usually upstream to the coding sequence of a structural gene, controls the expression of the coding region by providing the recognition for NA polymerase and/or other elements required for transcription to start at the correct site.

As used herein, the term “subject” refers to any vertebrate animal, including, but not limited to humans and other primates, rodents (e.g., mice, rats, and guinea pigs), lagamorphs (e.g., rabbits), bovines (e.g., cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.), as well as feral or wild animals, including, but not limited to, such animals as ungulates (e.g., deer), bear, fish, lagamorphs, rodents, birds, etc.

As used herein, a “transfected” cell is one into which an exogenous or heterologous nucleic acid sequence has been introduced. The nucleic acid sequence which has been introduced can be integrated into the genome of the transfected cell, or can be maintained episomally. A stably transfected cell is one in which the introduced DNA has integrated into a chromosome so that it is inherited by daughter cells through chromosome replication.

The term “vaccine” as used herein is defined as a material used to provoke an immune response after administration of the material to a vertebrate, typically a mammal. In certain embodiments a vaccine can be an immunogenic composition providing or aiding in prevention of disease. In other embodiments, a vaccine is a composition that can provide or aid in a cure of a disease. In others, a vaccine composition can provide or aid in amelioration of a disease. Further embodiments of a vaccine immunogenic composition can be used as therapeutic and/or prophylactic agents.

A “vector,” as used herein, refers to a replicon, such as plasmid, phage or cosmid, to which another DNA segment may be attached so as to bring about the replication of the attached segment.

The term “virus” as used herein is defined as a particle consisting of nucleic acid (RNA or DNA) enclosed in a protein coat, with or without an outer lipid envelope, which is capable of replicating within a whole cell.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### Description

Provided are methods for enhancing an antigen-induced immune response in a host comprising administering to a host an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen that is not expressed by the rabies virus, or a nucleic acid molecule encoding a Coronavirus antigen. The corresponding compositions, vaccines and kits are also provided.

Provided are compositions for enhancing an antigen-induced immune response in a host comprising administering to a host an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen that is not expressed by the rabies virus, or a nucleic acid molecule encoding a Coronavirus antigen.

The Rabies Virus (RV) for use in the practice of the present invention is attenuated. The modified G gene provides for a non-pathogenic rabies virus that eliminates or resists subsequent mutation resulting in a change of amino acids in the expressed glycoprotein from occurring. Thus, reversion to a pathogenic form is eliminated.

RV is a non-segmented negative-strand RNA virus within the Rhabdoviridae family and lyssavirus genera. The RV genome is about 12-kb in size and encodes five monocistronic RNAs encoding the nucleocapsid protein (N), phosphoprotein (P), matrix protein (M), the transmembrane glycoprotein (G), and the viral polymerase (L). The RV N protein encapsidates the viral RNA to form the ribonucleoprotein (RNP), which is the template for RNA transcription and replication by the viral polymerase-complex composed of the P and L proteins. The RV M bridges the RNP with the cytoplasmic domain (CD) of RV G in the host cell-derived viral membrane. The RV G mediates infection of the host cell. The main feature of rabies virus is neuroinvasiveness, which refers to its unique ability to invade the central nervous system (CNS) from peripheral sites.

RV is a promising vaccine vector able to induce humoral and cellular immune responses efficiently to foreign antigens. Recombinant live-viral vectors expressing foreign antigens efficiently induce potent cellular and humoral immune responses against the expressed antigens. Because of low seroprevalence in the human population, RV is an excellent viral vector candidate. Methods for engineering the virus are well established, up to two foreign genes totaling 6.5 kb have been incorporated thus far, and foreign sequences are stably maintained. RV grows to high titers in cell lines approved for human vaccine production and manufacture is economical. See, Smith et al, 2006, Virology, 353(2): 344-356. For example, replication-competent RV comprising heterologous nucleic acids sequences encoding the HIV-1 gpl 60 is described in WO 01/55330. Immunization with RV encoding bacterial, viral or cancer antigens, fused to at least a portion of the RV N protein or G protein is described in US Pat. Pub. 2008/0311147. Expression of HIV-1 Env or Gag results in potent immune responses directed against HIV-1 (Schnell et al., 2000, Proc. Natl. Acad. Sci USA 97(7): 3544-3549).

The availability of reverse genetics technology, has allowed the modification of RV viral elements that account for pathogenicity and immunogenicity, and has made the systematic development of safer and more potent modified- rabies vector feasible. For example, the pathogenicity of fixed RV strains (i.e., ERA, SAD) can be completely abolished for immunocompetent mice by introducing single amino acid exchanges in their G protein (Faber et al, 2005, J Virol 79: 14141-14148). RVs containing a SADB 19 G with an Arg₃₃₃ → Glu₃₃₃ mutation are nonpathogenic for adult mice after intracranial/intracerebral inoculation; an Asn₁₉₄→ Ser₁₉₄ mutation in the same gene prevents the reversion to pathogenic phenotype (Faber et al., 2005, J Virol 79: 14141-14148; Dietzschold et al., 2004, Vaccine 23:518-524; US Pat. 7,695,724). The G gene containing both mutations has been designated as “GAS”. The nucleotide sequence of the GAS gene is provided in US Pat. 7,695,724 as SEQ ID NO: 5, incorporated herein by reference. Using the GAS gene, the single and double GAS RV variants, SPBNGAS and SPBNGAS-GAS, respectively, were constructed (Faber et al., 2005, J Virol 79: 14141-14148; Li et al., 2008, Vaccine 26:419-426). The introduction of a second G gene significantly improves the efficacy of the vaccine by enhancing its immunogenicity through higher expression of G (Faber et al., 2002, J Virol 76:3374-3381). Elevated G expression is associated with the strong up-regulation of genes related to the NFKB signaling pathway, including IFN-α/β and IFN-y (Li et al., 2008, Vaccine 26:419-426) and increased cell death (Faber et al, 2002, J Virol 76:3374-3381).

Furthermore, the presence of two G genes also decreases substantially the probability of reversion to pathogenicity because the nonpathogenic phenotype determined by GAS is dominant over a pathogenic G that could emerge during virus growth in vivo or in vitro (Faber et al., 2007, J Virol 81 :7041-7047). A further improvement in recombinant RV safety is the highly attenuated triple RV G variant, SPBAANGAS-GAS-GAS (Faber et al., 2009, Proc. Natl. Acad. Sci USA 206(27): 1 1300-11305). The SPBAANGAS-GAS-GAS, also referred to herein as “TriGAS,” variant is completely nonpathogenic after intracranial infection of mice that are either developmentally immunocompromised (e.g., 5-day-old mice) or mice that have inherited deficits in immune function. The nucleotide sequence of the GAS gene is provided in US Pat. 7,695,724 as SEQ ID NO: 5. Attenuated rabies viruses have been engineered to express foreign antigens in the context of the virus particle, generally with limited success in terms of inducing immunity to the foreign antigen.

In one aspect, the present compositions and methods utilize the recombinant TriGAS virus. The TriGAS virus is a rabies virus expressing three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen. The TriGAS virus has been described in U.S. Pat. No. 8,282,939, which is incorporated herein by reference in its entirety. Compositions and methods utilizing the TriGAS virus has also been described in PCT Publication No. WO2014189654, which is incorporated herein by reference in its entirety.

The pathogenicity of RV strains can be completely abolished by mutating the glycoprotein so that the glycoprotein amino acid 194 is serine and the glycoprotein amino acid 333 is glutamic acid completely abolishes pathogenicity of rabies virus strains for immunocompetent mice. Rabies viruses containing a G having a mutation of Arg333 → Glu333 are non-pathogenic for adult mice after intracranial/intracerebral (i.e.) inoculation, and a mutation of Asn194 →Ser194 in the same gene prevents the reversion to pathogenic phenotype. The G gene containing both mutations is designated as GAS. GAS has been described in U.S. Pat. No. 7,695,724, which is incorporated herein by reference in its entirety. The nucleotide sequence of the GAS gene is provided in Pat. No. 7,695,724 as SEQ ID NO: 5 therein.

Because the attenuated TriGAS of the invention is not modified to express a foreign antigen, its tropism is unaffected and its exceptional safety profile is unchanged. Attenuated rabies viruses have been engineered to express foreign antigens in the context of the virus particle, generally with limited success in terms of inducing immunity to the foreign antigen. Recombinant approaches to alter the rabies virus genome and virus structure possess added risks in potentially altering tropism as well as causing cytopathic effects on infected cells that are not seen with TriGAS.

According to the present invention, the TriGAS virus functions to immunize a subject against rabies virus, and also to stimulate the immune system such that the immune response to a co-administered or separately administered Coronavirus antigen is enhanced. Thus, the TriGAS virus is both an immunogen, capable of safely immunizing a host against rabies virus, and also an immunostimulant/adjuvant for augmenting and enhancing the host immune response to a Coronavirus antigen. For example, efficacy of TriGAS as an immunostimulant has been shown, in the context of mixes of TriGAS and heterologous antigens, such as ovalbumin, Nipah virus glycoprotein, and others (see for example PCT Publication No. WO2014189654, which is incorporated herein by reference in its entirety).

In certain embodiments, the TriGAS virus is replication-competent. In certain embodiments, the composition comprises an inactivated or killed TriGAS RV. Inactivated/killed generally refers to infectious agents (e.g., bacteria, viruses, other microorganisms or agents) that are not capable of reproducing or cuaisng a productive infection. The inactivated/killed agents are able to stimulate an immune response when administered to a subject, in the context of a vaccine composition, for example. In contrast to inactivated vaccines, live vaccines and live attenuated vaccines, for example, are able to replicate and generally do so once they are administered to a subject.

Generally, agents for inclusion in an inactivated or killed vaccine may be grown, purified or semi-purified, inactivated, and then formulated into a vaccine composition. The virus may be killed or inactivated using a variety of methods. In one example, the virus may be treated with various chemicals for various periods of time to render the agents incapable of replication, but still retaining at least some ability to stimulate an immune response (i.e., immunogenicity) when administered to a subject. Many such agents are known. Example inactivating agents include, but are not limited to formalin/formaldehyde, ethyleneimine derivatives, ultraviolet radiation or heat, thimerosal and/or β-propiolactone, and others. The virus is generally treated with a concentration of the agent for a length of time and at a temperature to inactivate the virus, yet still preserve at least some of the ability of the agent to be immunogenic and stimulate an immune response. Inactivating agents may be removed prior to formulation into compositions for administration.

In certain embodiments, the TriGAS virus need not be replication competent to have the desired adjuvant effect. Inactivated/killed rabies viruses are exceptionally immunogenic, as is TriGAS. Thus, in certain embodiments, merely mixing a SAR-CoV-2 antigen (e.g., S protein or fragment thereof) mixed with inactivated TriGAS is sufficient to engender protective immunity to both SAR-CoV-2 and rabies virus. In certain instances there are possible immunological benefits to using inactivated virus in its generation of a different class (type 2) of immunity to a live virus (type 1). In certain embodiments, when using an inactivated virus (e.g. inactivated by UV treatment or beta-propiolactone treatment) a higher titer would be used in a mix (e.g., 10⁷ and higher), compared to replication-competent virus.

The subject may comprise a vertebrate. In one embodiment, the subject is a mammal. In one embodiment, the subject is a human being. Subjects for immunization according to the present invention include, for example, humans and other primates, rodents (e.g., mice, rats, and guinea pigs), lagamorphs (e.g., rabbits), bovines (e.g, cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.).

In one embodiment, the composition comprises a Coronavirus antigen or a nucleic acid molecule encoding a Coronavirus antigen. In one embodiment, the Coronavirus antigen is an antigen that may be a target for immune response that will result in treating or preventing disease caused by disease causing agents. In these embodiments, the Coronavirus antigen may comprise an antigen for which immunity is sought or against which antibodies can be raised.

In certain embodiments, the Coronavirus antigen is an antigen of any type of Coronavirus. Exemplary Coronaviruses include, but is not limited to, SARS-CoV-2, SARS-CoV, MERS-CoV, 229E, NL63, OC43, and HKU1. In one embodiment, the Coronavirus antigen comprises a SARS-CoV-2 antigen.

The disease causing agent or disease state has associated with it an antigen which triggers immune recognition and ultimate elimination or control of the disease causing agent or disease state in a host, by initiating a humoral and/or cellular immune response against the associated disease causing agent. Disease causing agents include but are not limited to pathogenic microorganisms. For example, in one embodiment, the disease causing agent is SARS-CoV-2, where SARS-CoV-2 infection causes COVID-19.

In another embodiment, the compositions and methods of the present invention are for vaccination against a disease causing agent in which the agent is a pathogenic microorganism. The pathogenic microorganisms may comprise a virus, such as a Coronavirus. The antigen thus comprises a Coronavirus antigen.

Disease causing virus agents include, for example, the following, wherein antigens provided are intended to be exemplary, not limiting: coronavirus, including but not limited to SARS-CoV-2, SARS-CoV, MERS-CoV, 229E, NL63, OC43, and HKU1. The Coronavirus antigen may comprise, for example, virus surface proteins, glycoproteins, or other virus protein antigens.

In one embodiment, the viral antigen is an antigen of a Coronavirus. The Coronavirus antigen can comprise any Coronavirus protein, Coronavirus protein subunit, Coronavirus protein domain, Coronavirus protein subdomain, or Coronavirus protein fragment.

In one embodiment, the viral antigen is an antigen of SARS-CoV-2. The SARS-CoV-2 antigen can comprise any SARS-CoV-2 protein, SARS-CoV-2 protein subunit, SARS-CoV-2 protein domain, SARS-CoV-2 protein subdomain, or SARS CoV-2 protein fragment. For example, in certain embodiments, the SARS-CoV-2 antigen comprises SARS-CoV-2 spike (S) glycoprotein, a subunit of S, or a peptide fragment of S. In certain embodiments, the SARS-CoV-2 antigen comprises a peptide fragment of S derived from the region of S that binds to angiotensin-converting enzyme 2, the putative cell receptor. Thus, in certain embodiments, the SARS-CoV-2 antigen comprises a peptide fragment of S that binds to angiotensin-converting enzyme 2. For example, in certain embodiments, the Tri-GAS acts as an adjuvant to induce a response to SARS-CoV-2 S antigens, causing a strong humoral response with minimal inflammation.

In certain embodiments, the composition comprises a peptide or polypeptide (e.g., a Coronavirus antigen) comprising an amino acid sequence that is substantially homologous to the amino acid sequence of a Coronavirus antigen, described herein and retains the function of the original amino acid sequence. For example, in certain embodiments, the amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, the composition comprises a peptide having one or more, two or more, three or more, four or more, five or more, six or more, seven or more, eight or more, nine or more, or ten or more mutations, such as point mutations, with respect to an amino acid sequence of a Coronavirus antigen, described herein.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer. The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, for example different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. For example, the identity between two amino acid sequences can be determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one aspect, the present invention provides a composition comprising an isolated nucleic acid molecule encoding one or more of the peptides or polypeptides described herein. For example, in certain aspects, the composition comprises DNA, RNA, mRNA, or cDNA encoding one or more of the peptides or polypeptides described herein.

In one embodiment, the composition comprises one or more isolated nucleic acid molecules encoding one or more Coronavirus antigens.

Further, the invention encompasses an isolated nucleic acid encoding an amino acid sequence having substantial identity to an amino acid sequence disclosed herein. In certain embodiments, the isolated nucleic acid sequence encodes an amino acid sequence that has at least 75%, 80%, 85%, 90%, 95%, 96%, 97%, 98%, or 99% sequence identity with an amino acid sequence disclosed herein.

The isolated nucleic acid sequence encoding a polypeptide of the invention can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a polypeptide of the invention, or functional fragment thereof. In one embodiment, the composition comprises an isolated RNA molecule encoding the polypeptide of the invention, or a functional fragment thereof.

The present invention also includes a vector in which the isolated nucleic acid of the present invention is inserted. The art is replete with suitable vectors that are useful in the present invention.

In brief summary, the expression of nucleic acids encoding a peptide of the invention is typically achieved by operably linking a nucleic acid encoding the peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno- associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the isolated nucleic acid encoding a polypeptide of the invention comprises in vitro transcribed (IVT) RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

In one aspect, the present invention provides a method of treating or preventing Coronavirus in a subject in need thereof. In one embodiment, the method comprises administering to the subject an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen , or a nucleic acid molecule encoding a Coronavirus antigen, wherein the Coronavirus antigen is not expressed by the rabies virus. In one embodiment, the subject has a Coronavirus infection. In one embodiment, the subject is at risk for having a Coronavirus infection.

In one embodiment, the method comprises treating or preventing a disease or disorder caused by a Coronavirus infection, comprising administering to the subject an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a Coronavirus antigen , or a nucleic acid molecule encoding a Coronavirus antigen, wherein the Coronavirus antigen is not expressed by the rabies virus. For example, in certain aspects, the method treats or prevents COVID-19, SARS, MERS, or other Coronavirus-associated disease or disorder. In one embodiment, the method comprises treating or preventing COVID-19 in a subject comprising administering to the subject an effective amount of: (a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and (b) a SARS-CoV-2 antigen, or a nucleic acid molecule encoding a SARS-CoV-2 antigen, wherein the SARS-CoV-2 antigen is not expressed by the rabies virus. In one embodiment, the subject has COVID-19. In one embodiment, the subject is at risk for having COVID-19.

Direct delivery of pharmaceutical compositions containing the nonpathogenic recombinant mutated TriGAS RV and/or Coronavirus antigen or nucleic acid molecule encoding a Coronavirus antigen in vivo may be accomplished via any route that provides immunity. In this regard, the compositions can be injected either subcutaneously, epidermaly, intradermally, intrathecally, intraorbitally, intramucosally (e.g., nasally, rectally and vaginally), intraperitoneally, intravenously, orally, or intramuscularly. Other modes of administration include oral and pulmonary administration, suppositories, and transdermal applications. In one embodiment, administration is by means of injection using a conventional syringe, or in the case of free-roaming wildlife, by hyperdermic darting.

The nonpathogenic recombinant mutated TriGAS RV and Coronavirus antigen or nucleic acid molecule encoding a Coronavirus antigen may be formulated in various pharmaceutical compositions, either separately or in combination. The compositions may include a pharmaceutically acceptable carrier and, optionally, can include other medicinal agents, pharmaceutical agents, carriers, adjuvants, diluents, and excipients. “Pharmaceutically acceptable” means a material that is not biologically or otherwise undesirable, i.e., the material can be administered to an individual along with the Coronavirus antigen or nucleic acid molecule encoding a Coronavirus antigen and/or recombinant RV without causing any undesirable biological effects or interacting in a deleterious manner with any of the other components of the pharmaceutical composition in which it is contained. Examples of physiologically acceptable carriers include saline solutions such as normal saline, Ringer’s solution, PBS (phosphate-buffered saline), and generally mixtures of various salts including potassium and phosphate salts with or without sugar additives such as glucose. Suitable excipients are, for example, water, saline, dextrose, glycerol, and ethanol. Nontoxic auxiliary substances, such as wetting agents, buffers, or emulsifiers may also be added to the composition. In one embodiment, adjuvants are not utilized for immunizations with the compositions of the invention.

Parenteral administration, if used, is generally characterized by injection. Sterile injectables can be prepared in conventional forms, either as liquid solutions or suspensions, solid forms suitable for solution or suspension- in liquid prior to injection, or as emulsions.

In certain embodiments, the nonpathogenic recombinant mutated TriGAS RV and the Coronavirus antigen, or nucleic acid molecule encoding a Coronavirus antigen, are administered simultaneously.

The nonpathogenic recombinant mutated TriGAS RV and the Coronavirus antigen, or nucleic acid molecule encoding a Coronavirus antigen, may be administered in the same composition or in different compositions. In certain embodiments, the nonpathogenic recombinant mutated TriGAS RV and the Coronavirus antigen, or nucleic acid molecule encoding a Coronavirus antigen, are administered in the same composition.

According to the invention, an immunologically effective amount of the compositions of the invention is administered to the subject in need of protection against a pathogen-induced disease in order to induce a protective immune response to the foreign protein antigen. An effective immunizing amount given to the subject is one in which a sufficient immunological response to the antigen is attained to provide a medically effective long-lasting immune response to the pathogen (e.g., SARS-CoV-2). For each recipient, the total vaccine amount to be administered can be deduced from protocols for immunization with other vaccines. The exact amount of nonpathogenic recombinant mutated TriGAS RV and Coronavirus antigen, or nucleic acid molecule encoding a Coronavirus antigen, will vary from subject to subject, depending on the species, age, weight and general condition of the subject, the particular strain of RV and the nature of the Coronavirus antigen, mode of administration, and the like. The immunologically effective dosage or the effective immunizing amount that inoculates the animal and elicits satisfactory immune response can be easily determined or readily titrated by routine testing such as, for example, by standard dose titration studies. Generally, dosage will approximate that which is typical for the administration of other vaccines. In certain embodiments, a single dose of nonpathogenic recombinant mutated TriGAS RV administered to the subject is from about 10⁴ to about 10⁶ tissue culture infectious units (TCIU), or from about 10⁵ to about 10⁶ TCIU. In certain embodiments, where inactivated TriGAS RV is utilized, a higher titer (e.g., 10⁷ and higher) is used. Multiple dosing is also contemplated.

In some embodiments, following a prime immunization with the nonpathogenic recombinant mutated TriGAS RV and Coronavirus antigen, or nucleic acid molecule encoding a Coronavirus antigen, the subject is inoculated at least once with a booster composition containing the Coronavirus antigen or nucleic acid molecule encoding a Coronavirus antigen. The booster composition comprises the non-rabies antigen in a pharmaceutically acceptable carrier. The booster composition may utilize any vehicle suitable for parenteral administration of the Coronavirus antigen or nucleic acid molecule encoding a Coronavirus antigen. The booster composition may contain the carrier substances noted above for formulation of the nonpathogenic recombinant mutated TriGAS RV. In one embodiment, the booster composition may be adjuvant-free.

The booster composition may comprise, phosphate- buffered saline, aqueous sodium chloride, e.g., a 0.9% sodium chloride solution. In one embodiment, the booster composition is dispersed or dissolved in the vehicle providing the booster composition. In one embodiment, the non-rabies antigen is water soluble and is dissolved in an aqueous-based adjuvant-free vehicle, such as saline solution or phosphate- buffered saline. The booster composition may comprise additional ingredients such as stabilizers or other formulation aids, provided that it does not contain adjuvant.

The booster immunization may be administered following initiation of an immune response to the non-rabies antigen by the compositions of the invention. Generally, at least about 5 days, or at least about 10 days, should be permitted to lapse following the primary immunization with the composition of the invention before boosting. In some embodiments, booster immunization occurs within 1 to 300 days, 5 to 250, 10 to 200, 15 to 150, 20 to 100, or 30 to 60 days following primary immunization with recombinant RV and the Coronavirus antigen, or nucleic acid molecule encoding a Coronavirus antigen. In specific embodiments, a booster immunization is administered 10, 1 1 , 12, 13, 14, 15, 16, 17, 18, 19, 20, 21 , 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, 57, 58, 59 or 60 days following primary immunization.

Multiple booster immunizations may be administered to augment the host immune response to the foreign protein antigen. For example, 2, 3, 4, 5, or 6 booster immunizations may be administered in intervals. The intervals between booster immunizations may be uniform, e.g., booster immunizations are spaced apart by 5, 10, 20, 25, or 30 day intervals, or the booster immunizations may be scheduled at irregular time intervals.

The progress of the subject’s immune response to the Coronavirus antigen may be monitored by conventional assay methods. For example, the appearance of antibodies to the antigen in the blood or serum of the inoculated subject may be monitored by standard antibody assay methods including, but not limited to, radioimmunoassay, ELISA (enzyme- linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and Immunoelectrophoresis assays.

In one embodiment, a kit is provided comprising two vaccine preparations suitable for parenteral administration. The kit comprises a first composition comprising nonpathogenic recombinant mutated TriGAS RV and at least one Coronavirus antigen of interest, or at least one nucleic acid molecule encoding at least one Coronavirus antigen, for immunizing a subject. The kit further comprises a second composition for boosting the immune response of the subject to the Coronavirus antigen. In certain embodiments, the first composition or second composition optionally contain adjuvant. In certain embodiments, the first composition or second composition is adjuvant -free. The respective compositions may be in liquid or solid (lyophilized) form.

The kits of the present invention may optionally comprise different containers (e.g., vial, ampoule, test tube, flask or bottle) for each individual composition. The kit may contain additional reagents, such as buffers, diluents and the like, for formulation the individual components. Each component will generally be suitable as aliquoted in its respective container or provided in a concentrated form.

Instructions for using the kit according to the immunization methods described above may be included. The instructional material may comprise a publication, a recording, a diagram, or any other medium of expression which can be used to communicate directions for use of the kit. A package insert may comprise text housed in any physical medium, e.g., paper, cardboard, film, or may be housed in an electronic medium such as a diskette, chip, memory stick or other electronic storage form. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

In further embodiments, the Coronavirus antigen is administered conjugated to a carrier, such as keyhole limpet hemocyanin (KLH).

It has been shown that KLH is safe to administer as a carrier protein (Helling, F. et al, 1995, Cancer Res. 5S:2783-2788, U.S. Pat. No. 5,102,663). KLH or a derivative thereof may be conjugated to an antigen directly, or via a linker. An example of a linker that may be used to conjugate KLH to an antigen is maleimidobenzoyl-N-hydroxy-succinimide ester (MBS).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Immunization With TriGAS and OVA

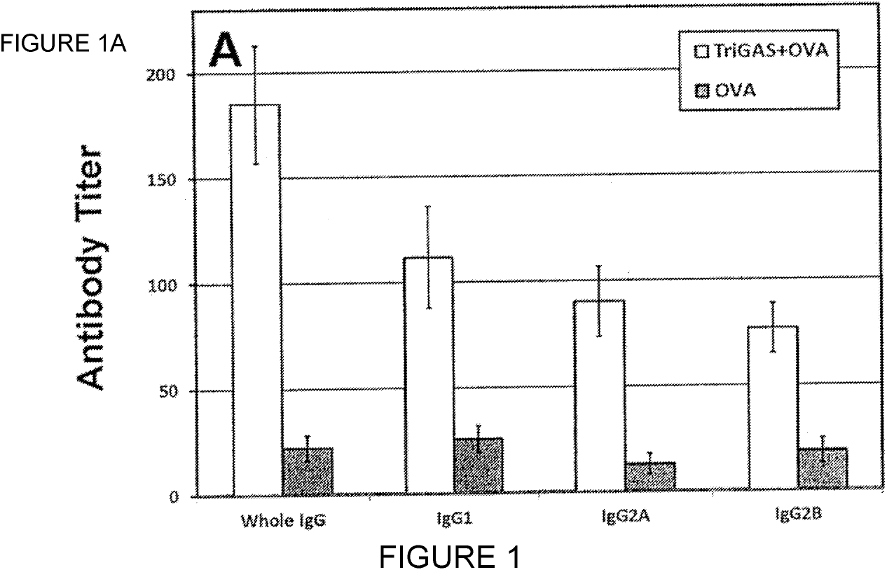

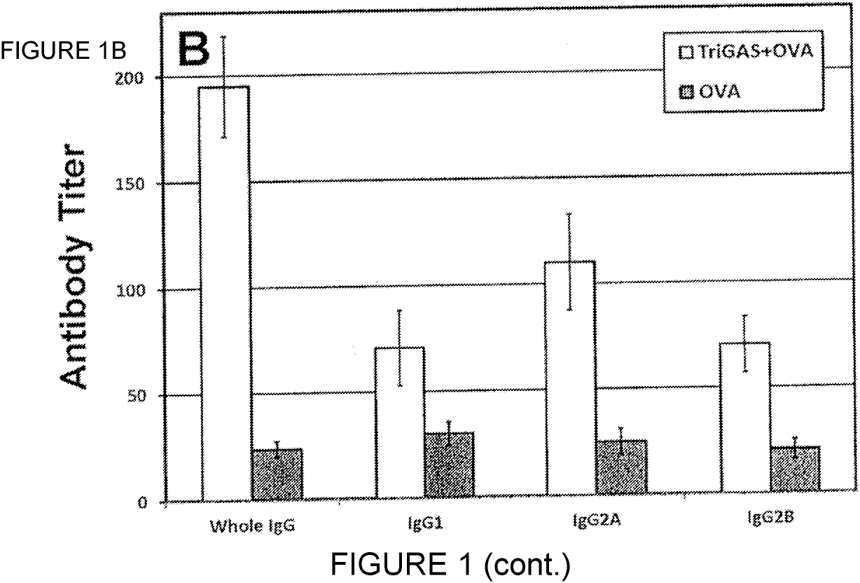

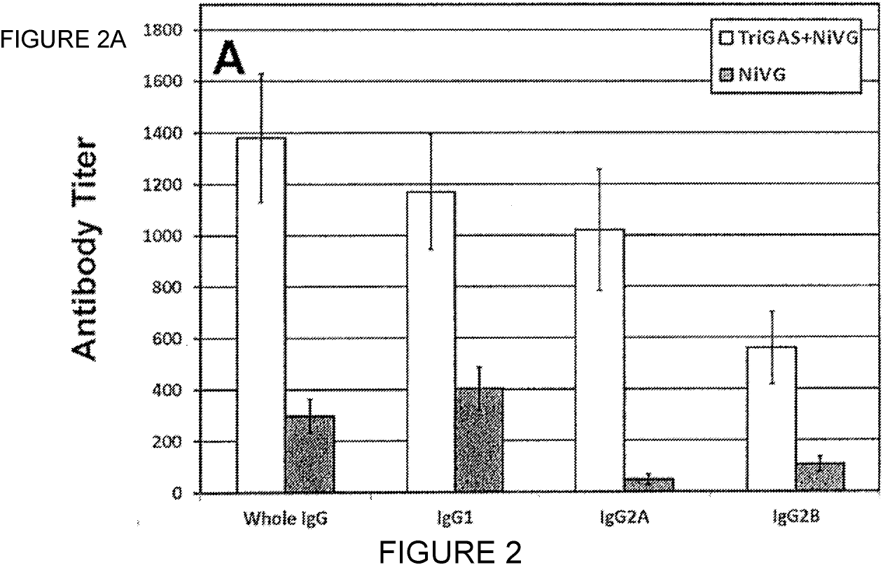

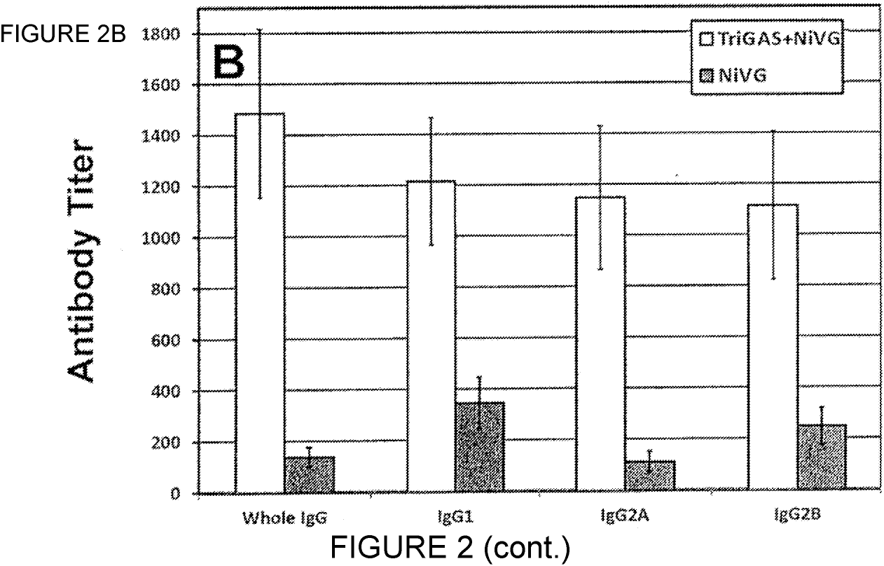

Production of ovalbumin (OVA)-specific antibody after immunization with a single dose of a mixture of nonpathogenic recombinant mutated TriGAS (“TriGAS”) and OVA or OVA alone was studied. Groups of ten Swiss Webster mice were immunized intramuscularly (i.m.) with 100 µL PBS containing 10⁶ FFU TriGAS and 50 µg OVA (open bars) or 100 µL PBS containing only 50 µg OVA (closed bars). The mice were bled 21 days (FIG. 1A) and 2 months (FIG. 1B) after immunization and the isotypes of OVA-specific antibody were determined by ELISA using alkaline-phosphatase conjugated anti-mouse whole IgG or biotinylated anti-mouse IgG1, IgG2a, or lgG2b. The results are illustrated in FIG. 1A and FIG. 1B. The antibody titers are presented as mean titers (+/- Standard Error) calculated from ten mice per group.

FIG. 1A and FIG. 1B illustrate that while an administration of a single dose of OVA in the absence of TriGAS was poorly or not immunogenic, the mixture of OVA with TriGAS induced a strong immune response to the OVA, as measured by antibody production. Most importantly, the anti-OVA immune response induced by the OVA-TriGAS mixture is longlasting (FIG. 1B).

### Example 2: Immunization With TriGAS and NiVG

Production of NiVG-specific antibody after immunization with a single dose of a mixture of TriGAS and NiVG or NiVG alone was studied. Groups of ten Swiss Webster mice were immunized intramuscularly (i.m.) with 100 µi PBS containing 10⁶ FFU TriGAS and 12.5 µg NiVG (open bars) or 100 µi̅ PBS containing only 12.5 µg NiVG (closed bars). The mice were bled 21 days (FIG. 2A) and 2 months (FIG. 2B) after immunization and the isotypes of NiVG-specific antibody were determined by ELISA using alkaline-phosphatase conjugated anti-mouse whole IgG or biotinylated anti-mouse IgG1, IgG2a, or IgG2b. The results are illustrated in FIGS. 2A and 2B. The antibody titers are presented as mean titers (+/- Standard Error) calculated from 0 mice per group.

FIG. 2A and FIG. 2B illustrate that while an administration of a single dose of NiVG in the absence of TriGAS was poorly or not immunogenic, the mixture of NiVG with TriGAS induced a strong, long-lasting (FIG. 2B) immune response to the NiVG, as measured by antibody production.

### Example 3: Immunization With TriGAS and GnRH-KLH or With GnRH-KLH Alone

Production of GnRH-specific antibody after immunization with a single dose of a mixture of TriGAS and GnRH-keyhole limpet hemocyanin (KLH) or with a single dose of GnRH-KLH alone was studied. Groups of ten Swiss Webster mice were immunized intramuscularly (i.m.) with 100 µL PBS containing 10⁶ FFu TriGAS and 50 µg GnRH (as GnRH-KLH), or 100 mL PBS containing only GnRH-KLH (50 µg GnRH). The mice were bled 1 days, 3 months, and 6 months after immunization and GnRH-specific antibody were determined by ELISA using alkaline phosphatase-labelled anti-mouse whole IgG. The antibody titers are presented as mean titers (+/- Standard Error) calculated from ten mice per group.

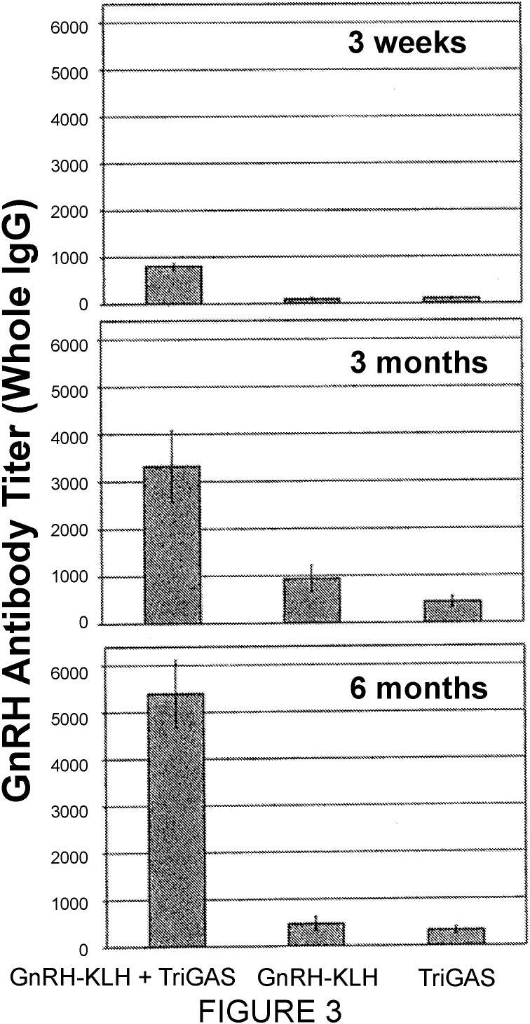

FIG. 3 illustrates that while an administration of a single dose of GnRH-KLH in the absence of TriGAS was poorly or not immunogenic, the mixture of TriGAS and GnRH-KLH induced a strong, long-lasting antibody response to GnRH (whole IgG) which actually increased over time.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of inducing an immune response against a coronavirus in a subject comprising administering to the subject an effective amount of:
(a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and
(b) a Coronavirus antigen, or a nucleic acid molecule encoding a Coronavirus antigen, wherein the Coronavirus antigen is not expressed by the rabies virus.

2. The method according to claim 1 wherein the Coronavirus antigen comprises an antigen of a Coronavirus selected from the group consisting of: SARS-CoV-2, SARS-CoV, MERS-CoV, 229E, NL63, OC43, and HKU1.

3. The method according to claim 1, wherein the Coronavirus antigen comprises SARS-CoV-2 Spike (S) protein, or fragment thereof.

4. The method of claim 1, wherein the Coronavirus antigen comprises a fragment of SARS-CoV-2 S that binds to angiotensin-converting enzyme 2.

5. The method according to claim 1 wherein the nonpathogenic recombinant rabies virus and the Coronavirus antigen, or nucleic acid molecule encoding the Coronavirus antigen, are administered simultaneously.

6. The method according to claim 1 wherein the nonpathogenic recombinant rabies virus is administered before the Coronavirus antigen, or nucleic acid molecule encoding the Coronavirus antigen.

7. The method according to claim 1 wherein the nonpathogenic recombinant rabies virus and the Coronavirus antigen, or nucleic acid molecule encoding the Coronavirus antigen, are administered in the same composition.

8. The method according to claim 1 wherein the nonpathogenic recombinant rabies virus and the Coronavirus antigen, or nucleic acid molecule encoding the Coronavirus antigen, are administered in a different composition.

9. The method of claim 1 wherein the nonpathogenic recombinant rabies virus and the Coronavirus antigen, or nucleic acid molecule encoding the Coronavirus antigen, are administered intranasally, subcutaneously, epidermaly, intradermally, intrathecally, intraorbitally, intramucosally, intraperitoneally, intravenously, orally, or intramuscularly.

10. A composition comprising:
(a) a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and
(b) a Coronavirus antigen, or a nucleic acid molecule encoding a Coronavirus antigen, wherein the Coronavirus antigen is not expressed by the rabies virus.

11. The composition according to claim 10, wherein the Coronavirus antigen comprises an antigen of a Coronavirus selected from the group consisting of: SARS-CoV-2, SARS-CoV, MERS-CoV, 229E, NL63, OC43, and HKU1.

12. The composition according to claim 10, wherein the Coronavirus antigen comprises SARS-CoV-2 Spike (S) protein, or fragment thereof.

13. The composition according to claim 10, wherein the Coronavirus antigen comprises a fragment of SARS-CoV-2 S that binds to angiotensin-converting enzyme 2.

14. A vaccine comprising the composition of claim 10.

15. A kit for immunization of a subject comprising:
(a) a first composition comprising a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and
(b) a Coronavirus antigen, or a nucleic acid molecule encoding a Coronavirus antigen, wherein the Coronavirus antigen is not expressed by the rabies virus.

16. The kit according to claim 15, wherein the Coronavirus antigen comprises an antigen of a Coronavirus selected from the group consisting of: SARS-CoV-2, SARS-CoV, MERS-CoV, 229E, NL63, OC43, and HKU1.

17. The kit according to claim 15, wherein the Coronavirus antigen comprises SARS-CoV-2 Spike (S) protein, or fragment thereof.

18. The kit according to claim 15, wherein the Coronavirus antigen comprises a fragment of SARS-CoV-2 S that binds to angiotensin-converting enzyme 2.

19. A method of immunizing a subject against rabies virus and against a Coronavirus antigen comprising the steps of:
(a) administering a first composition comprising a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and
(b) administering a second composition comprising a Coronavirus antigen, or a nucleic acid molecule encoding a Coronavirus antigen, wherein the Coronavirus antigen is not expressed by the rabies virus.

20. The method according to claim 19, wherein the Coronavirus antigen comprises an antigen of a Coronavirus selected from the group consisting of: SARS-CoV-2, SARS-CoV, MERS-CoV, 229E, NL63, OC43, and HKU1.

21. The method according to claim 19, wherein the Coronavirus antigen comprises SARS-CoV-2 Spike (S) protein, or fragment thereof.

22. The method of claim 19, wherein the Coronavirus antigen comprises a fragment of SARS-CoV-2 S that binds to angiotensin-converting enzyme 2.

23. A method of immunizing a subject against a Coronavirus antigen comprising the steps of:
(a) administering a first composition comprising a nonpathogenic recombinant rabies virus comprising at least three copies of a mutated G gene, wherein said mutated G gene encodes a rabies virus glycoprotein wherein the glycoprotein amino acid 194 is serine and the glycoprotein amino acid 333 is glutamic acid, wherein said recombinant rabies virus does not express a foreign protein antigen; and
(b) administering a second composition comprising a Coronavirus antigen, or a nucleic acid molecule encoding a Coronavirus antigen, wherein the Coronavirus antigen is not expressed by the rabies virus.

24. The method according to claim 23, wherein the Coronavirus antigen comprises an antigen of a Coronavirus selected from the group consisting of: SARS-CoV-2, SARS-CoV, MERS-CoV, 229E, NL63, OC43, and HKU1.

25. The method according to claim 23, wherein the Coronavirus antigen comprises SARS-CoV-2 Spike (S) protein, or fragment thereof.

26. The method of claim 23, wherein the Coronavirus antigen comprises a fragment of SARS-CoV-2 S that binds to angiotensin-converting enzyme 2.

27. A method comprising administering the composition of claim 10 to a subject.

28. The method of claim 27, wherein the subject has a severe acute respiratory syndrome coronavirus 2 (SARS-CoV-2).
